(19) United States
(12) Patent Application Publication
(10) Pub. No.: US 2005/0192487 A1
(43) Pub. Date: Sep. 1, 2005

(54) System for collection, manipulation, and analysis of data from remote health care devices

(76) Inventors: Louis C. Cosentino, Deephaven, MN (US); Daniel L. Cosentino, Chaska, MN (US); Todd F. Young, Cologne, MN (US)

Correspondence Address: Merchant & Gould P.C., P.O. Box 2903, Minneapolis, MN, 55402-0903, US

(21) Appl. No.: 10/788,900

(22) Filed: Feb. 27, 2004

(51) Int. Cl. A61B 5/00; A61B 10/00; G06F 17/60; G06F 17/00

(52) U.S. Cl. 600/300; 128/920; 705/2

(57) ABSTRACT

A system for determining whether a person should have professional health care attention, and providing clinical notes to the caregiver, may include the following. The system includes a monitoring device having a microprocessor operably coupled to a memory unit. The microprocessor is operably coupled to an input device, an output device, and a communication device. The memory unit is programmed with a set of instructions for posing questions to the person via the output device, receiving answers from the person via the input device, and transmitting the answers to a remote computer via the communication device. The remote computer is programmed to determine whether the person should be seen by a health care professional, based at least in part upon the answers entered into the input device. Further, the remote computer is programmed to generate a clinical note based upon the answers transmitted to the remote computer.

### TECHNICAL FIELD

The invention relates generally to a computerized system for collecting, manipulating, and analyzing data from a populace of remote health care devices, so that a sub-populace needing medical attention may be identified.

### BACKGROUND

As the cost of health care has increased, technologies have been developed to more efficiently deploy existing health care services. One such technology involves the deployment of devices that collect patient data and transmit that data to a data analysis center that is associated with one or more institutions, facilties, call centers, health and fitness clubs, or health care centers. The devices are typically given to a large populace of patients associated with a health care center. For example, a cardiac center may provide such devices to each of its patients. The patients may keep the devices in their homes. The devices typically collect data in two manners. The device asks questions aimed at determining whether or not the patient should have health care professional attention (e.g., the device asks questions that indicate whether or not a patient's heart condition is particularly severe on a given day). Additionally, the device may employ a biometric measurement unit. For example, the device may include a scale that weighs the patient to determine if fluid is collecting in the patient's lungs or extremities (when fluid collects in a patient's lungs, the patient's weight rises demonstrably).

Devices of the sort described above are made available by Cardiocom LLC, and are marketed under the trademarks TELESCALE®, CARESTAR™, and THINLINK™. These devices operate based upon a unique premise: the devices collect information at a cost that is far less than the economic value of the information they provide. For example, the devices are placed in patient homes at a cost. Based upon the information collected by the devices, patient hospitalization may be avoided by identifying particular patients that require a medication adjustment or should have health care professional attention before the particular patient's condition becomes so severe that hospitalization is needed. For this strategy to work, it is important that the information collected by the devices be processed intelligently, so that the proper sub-populace can be identified, so that the proper parties are notified when a patient needs assistance, and so that necessary information regarding a patient is available when decisions regarding the health care of a patient are being made.

For the foregoing reasons, it is evident that there exists a need for a computer system that addresses the above described needs in a simple-to-operate and cost effective manner to manage large patient populations.

### SUMMARY OF THE INVENTION

Against this backdrop the present invention was developed. A system addressing the aforementioned problems, including determining whether a person should have health care professional attention, and providing clinical notes to the caregiver, may include the following. The system may include a monitoring device having a microprocessor operably coupled to a memory unit. The microprocessor may also be operably coupled to an input device, an output device, and a communication device. The memory unit may be programmed with a set of instructions for posing questions to the person via the output device, receiving answers from the person via the input device, and transmitting the answers to a remote computer via the communication device. The remote computer may be programmed to determine whether the person should have health care professional attention, based at least in part upon the answers entered into the input device. Further, the remote computer may be programmed to generate a clinical note based upon the answers transmitted to the remote computer.

According to another embodiment, a computer system for interfacing with a monitoring device that poses questions regarding disease state symptoms to a person, receives answers from the person, and transmits the answers to the computer system, may include the following. The computer system may include a microprocessor operably coupled to a memory unit, an input device, an output device, and a communication device. The memory unit may be programmed with a set of instructions for determining whether the person should have health care professional attention based at least in part upon the answers transmitted to the computer system. The memory unit may be further programmed to generate a clinical note based upon the answers transmitted to the computer system.

According to yet another embodiment, a computerized method of interfacing with a monitoring device that poses questions regarding disease state symptoms to a person, receives answers from the person, and transmits the answers to the computer system may include the following acts. The method may include determining whether the person should have health care professional attention based at least in part upon the answers transmitted to the computer system. The method may also include generating a clinical note based upon the answers transmitted to the computer system.

According to yet another embodiment of the invention, a system determining whether a person should have health care professional attention, may include the following. The system may include a monitoring device having a microprocessor operably coupled to a memory unit. An input device, an output device, and a communication device may also be operably coupled to the microprocessor. The memory unit may be programmed with a set of instructions for posing questions to the person via the output device, receiving answers from the person via the input device, and transmitting the answers to a remote computer via the communication device. The remote computer may be programmed to determine whether the person should have health care professional attention based at least in part upon the answers entered into the input device. Further, the remote computer may be programmed to permit entry, storage, and presentation of intervention data.

According to another embodiment, a computer system for interfacing with a monitoring device that poses questions regarding disease state symptoms to a person, receives answers from the person, and transmits the answers to the computer system, may include the following. The computer system may include a microprocessor operably coupled to a memory unit, an input device, an output device, and a communication device. The memory unit may be programmed with a set of instructions for determining whether the person should have health care professional attention, based at least in part upon the answers entered into the input device. Further, the memory unit may be programmed with a set of instructions for permitting entry, storage, and presentation of intervention data.

According to yet another embodiment, a method, carried out by a computer system, of interfacing with a monitoring device that poses questions regarding disease state symptoms to a person, receives answers from the person, and transmits the answers to the computer system, may include the following. The method may include determining whether the person should have health care professional attention, based at least in part upon the answers transmitted to the computer system. The method may also include permitting entry, storage, and presentation of intervention data.

### BRIEF DESCRIPTION OF THE DRAWINGS

FIG. 1 depicts a computer system, according to one embodiment of the present invention.

FIG. 2 depicts a process flow, according to one embodiment of the present invention.

FIG. 3 depicts a skeletal view of a user interface, according to one embodiment of the present invention.

FIG. 4 depicts an example of an exception monitoring screen according to one embodiment of the present invention.

FIG. 5 depicts an example of a screen associated with a patient tab, according to one embodiment of the present invention.

FIG. 6 depicts an example of a screen associated with a medications tab, according to one embodiment of the present invention.

FIG. 7 depicts an example of a screen associated with a contacts tab, according to one embodiment of the present invention.

FIG. 8 depicts an example of a screen associated with a status tab, according to one embodiment of the present invention.

FIG. 9 depicts an example of a screen associated with a history tab, according to one embodiment of the present invention.

FIG. 10 depicts an example of a screen associated with a labs tab, according to one embodiment of the present invention.

FIG. 11 depicts an example of a screen associated with a notes tab, according to one embodiment of the present invention.

FIG. 12 depicts an example of a screen associated with a verify tab, according to one embodiment of the present invention.

FIG. 13 depicts an example of a screen associated with a setup tab, according to one embodiment of the present invention.

FIG. 14 depicts a weight graph screen, according to one embodiment of the present invention.

FIG. 15 depicts a symptoms graph screen, according to one embodiment of the present invention.

FIG. 16 depicts a clinical note builder in accordance with one embodiment of the present invention.

FIG. 17 depicts an expert system in accordance with one embodiment of the present invention.

### DETAILED DESCRIPTION

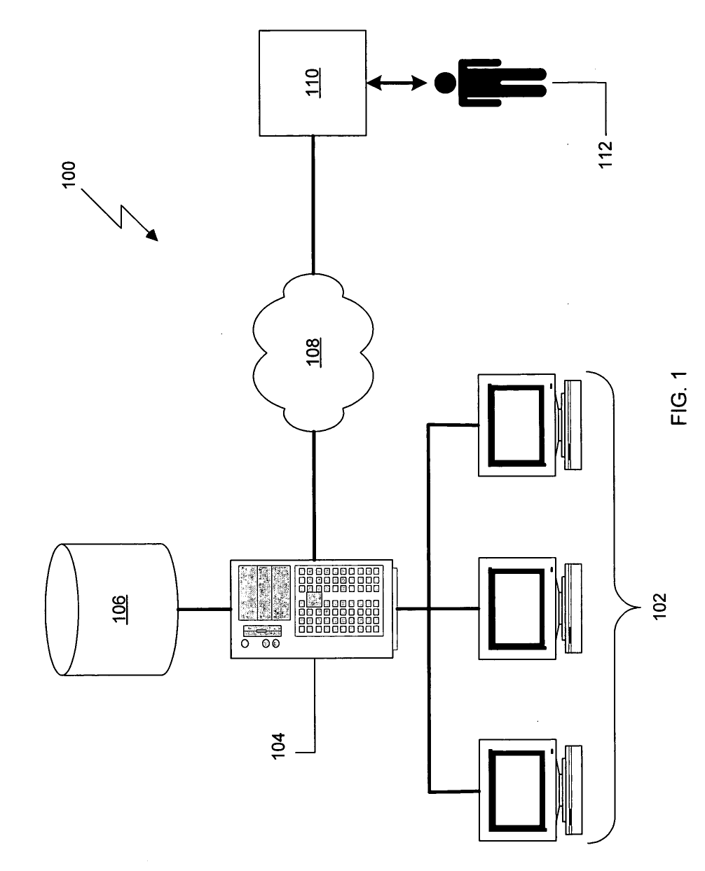

The system disclosed in FIGS. 1-17 ensures that an attendant, such as an operator at a call center, knows which users to call, why to call the users, and when to call the users. The attendants may be of various skill and educational levels. For example, an attendant may be an individual with no health care training, or may be a registered nurse. The system disclosed herein contains features that minimize the occassions upon which a person is called upon to manually interpret and process health care data from the user or the user's device. Thus, the number of skilled attendants employed by a health care center may be greatly reduced. For example, a call center may operate with 200 or 250 patients per nurse. Some of the features disclosed herein may boost that ratio to an even greater number of patients per nurse. Such a boost increases call center efficiency, and reduces the cost of health care for all.

The system described herein ensures that the users receive care that consistent with best practices or standard procedures that have been developed by the health care professional facility. Specifically, an expert system and related features described promote and ensure consistent interaction between the users and operators employed by the call center.

The user interface is uniquely designed for efficient, effect management of large patient populations using remote monitoring devices. The interface presents patient data in a sensible layout, and esnures that needed data can be obtained with a minimal number of button or mouse “clicks.” This unique layout improves the efficiency of remote monitoring.

FIG. 1 depicts a computer system 100 according to one embodiment of the present invention. The computer system 100 may be located in a call center associated with one or more facilities, institutions, health and fitness centers, or health care facilities or may be located within a health care facility, for example. Throughout the disclosure, the system 100 is referred to as though it were located in a call center, although this is not essential to the invention.

The system 100 includes one or more workstations 102, which may be accessed by one or more operators. The workstations 102 are of ordinary construction, and include a processor coupled to a bus. The bus couples the processor to one or more memory devices, peripherals, mass storage devices, communication devices, and the like. The workstations 102 are programmed to request data from a server 104, which is coupled to a datastore 106. The datastore 106 may be embodied as a database, but this is not essential. The workstations 102 present a user interface that permits operators to access data related to a patient populace or to a particular patient. The workstations 102 also permit an operator to enter data related to a patient, so that the data may be accessed at a future time.

The server 104 is connected via a network 108, such as a telephonic network or the Internet, to a device 110 which may be located in the home of a user 112. Although FIG. 1 depicts but a single user 112, the system 100 may be accessed by a plurality of users 112. For example, a cardiac center (not depicted) may employ a call center to operate the system 100. The cardiac center may provide devices 110 to each of its patients, and the devices 110 may be kept in the home of each of the patients.

In use, the system 100 operates as generally described below. The user 112 interacts with the device 110 on a regular basis (e.g., the user 112 interacts with his or her device 110 on a daily basis). The device 110 asks the patient a series of questions, in order to gather information from which it can be determined if the user 112 should have health care professional attention. The user 112 may be in need of medical attention for several reasons. For example, the user 112 may have a chronic condition that is acute on a given day. The questions are designed to identify the fact that the user's 112 condition is acute. Alternatively, the user's 112 condition may be changing in some material way, although the condition may not be acute. The change may indicate the onset of a complication that needs to be assessed by a health care professional. The questions are also designed to gather information from which it may be concluded that the user's 112 condition is changing in some material way.

The device 110 may also include a biometric measuring unit. A biometric measurement unit is a device that takes a measurement of a medically significant, objective variable. For example, the device 110 may include a scale to weigh the user 112. The device 110 may also include a blood glucose measurement unit, a pulse measurement unit, a blood pressure measurement unit, or any other biometric measurement unit. The measurement returned by the biometric measurement unit may also be determinative of whether the user 112 should have health care professional attention. Thus, the user's 112 interaction with the device 110 may also include permitting the biometric measurement unit to take one or more measurements (e.g., the user 112 may be instructed to step upon a scale so that the user's 112 weight may be measured).

After the user's 112 interaction with the device 110 is complete, the device 110 communicates with the server 104. The device 110 uploads the data collected from the user 112 (e.g., the device 110 uploads the answers to the questions posed and any biometric data gathered from the patient). The server 104 responds by storing the data in the datastore 106. The device 110 may also download data during the communication session. For example, the device 110 may download new questions to be asked to the patient on a subsequent day. Per one embodiment, the device 110 is preprogrammed with a set of questions. The set of questions may include subsets, each of which are directed toward a different theme. During the communication session, the device 110 may download data that indicates that certain of the subsets of questions of questions are to be activated or deactivated. Additionally, the device 110 may download verbiage to be posed to the user 112 (e.g., a question to be presented to the user 112 or a statement to be declared to the user 112) during the user's 112 subsequent interaction with the device 110.

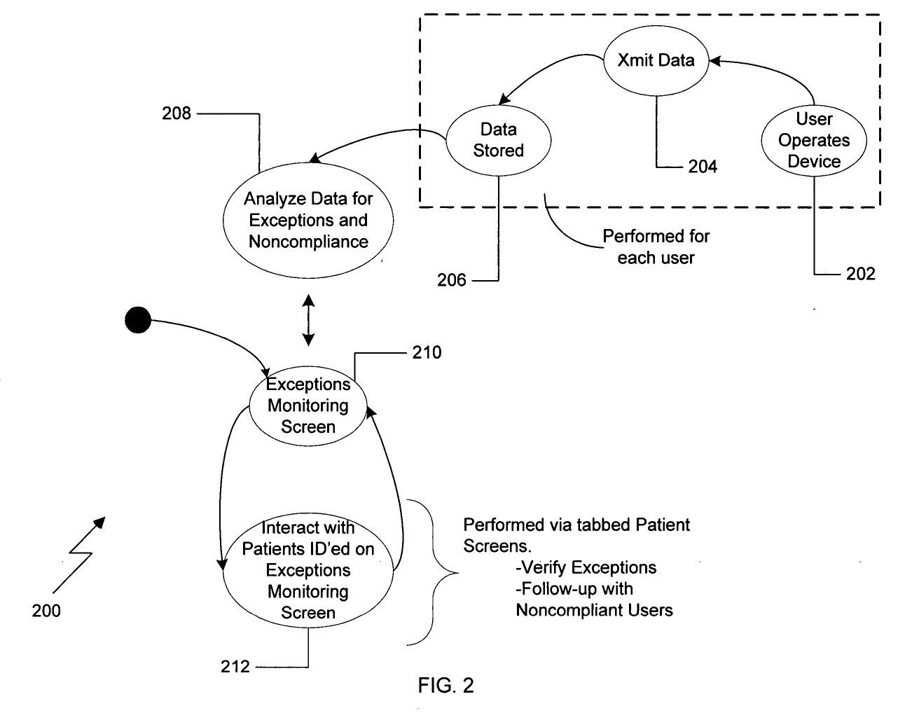

The steps just described are depicted in the system flow 200 shown in FIG. 2. As shown therein, each user 112 interacts with his or her device 110 on some regular basis, as shown in operation 202. As described above, the interaction 202 may include answering questions and/or submitting to a biometric measurement. Thereafter, the data obtained from the interaction 202 is transmitted to the server 104, as shown in operation 204. Of course, data may be downloaded during this operation, as described above. In response to having received data from a particular device 110, the server 104 stores the data in the datastore 106, as shown in operation 206. As indicated in FIG. 2, these operations are carried out for each device 110 deployed by the call center.

At a given point in time, the server 104 analyzes the data in the datastore 106 for the purpose of determining which users 112 seem to need health care professional attention, and to determine which users 112 failed to interact with their devices. For example, if the users 112 were instructed to interact with their devices by 11:00 AM, the server 104 may execute operation 208 at 12:00 PM, a point in time by which all user interaction was to have taken place. Alternatively, the server 104 may execute this operation (or any of the operations described herein) continuously or as data is received. The server may analyze the data for the purpose of declaring an “exception” with respect to certain users 112. An exception is a condition indicating that a user 112 appears to need contact with a medical professional for one reason or another. An exception may be declared in several ways, some of which are discussed generally, below. Thus, operation 208 divides the populace of users 112 into two sub-populaces on a given day: (1) users 112 for whom an exception was declared, and therefore need to be contacted that day; and (2) users 112 for whom an exception was not declared, and therefore do not need to be contacted that day.

As shown in FIG. 2, operators begin their interaction with their workstations 102 by being presented with an exception monitoring screen, as shown in operation 210. An exception monitoring screen is a screen that presents the operator with a list of users 112 who have been declared as having an exception and/or with a list of users that failed to user their device within the preceding day.

In response to being presented with the exception monitoring screen, the operators endeavor to contact each user 112 identified as having an exception or identified as not having operated their device within the last day (users 112 that have failed to user their device may be referred to as “noncompliant”). An operator may contact a user 112 having been identified as having an exception or as being noncompliant by placing a telephone call to that user 112, for example. Prior to interacting with the user 112, an operator may open a patient screen that pertains to the user with whom contact is to be made. For example, prior to placing a telephone call to a particular user 112, the operator may double-click the user's name on the exception monitoring screen. Double-clicking the user name causes a patient screen pertaining to the user identified by the double-clicked user name to be opened. During interaction with the user 112, the operator is available to review data pertaining to the user 112, to edit data pertaining to the use 112, or to record notes or impressions relating to the user 112.

The purpose of the interaction between the operator and the user 112 depends upon why the user is being contacted. If the user is being contacted because the user failed to use the device 110, the operator may simply remind the user 112 to interact with his or her device 110. If the user 112 were to inform the operator that the device is broken, the operator may schedule maintenance for the device. If, on the other hand, an operator is contacting a user 112 because the user is identified as having an exception, the operator may want to verify that the user in fact needs health care professional attention, a care plan or medication adjustment, or disease specific education. The telephone call may include verifying that the user 112 answered the questions correctly (if the user accidentally answered a question incorrectly, the operator may change the user's answers via the patient screen). The operator may also interview the user 112 to arrive at a preliminary analysis of the user's condition and to arrive at a preliminary corrective action. Such a preliminary analysis and conclusion may be recorded in the form of a note that may be subsequently communicated to a health care professional.

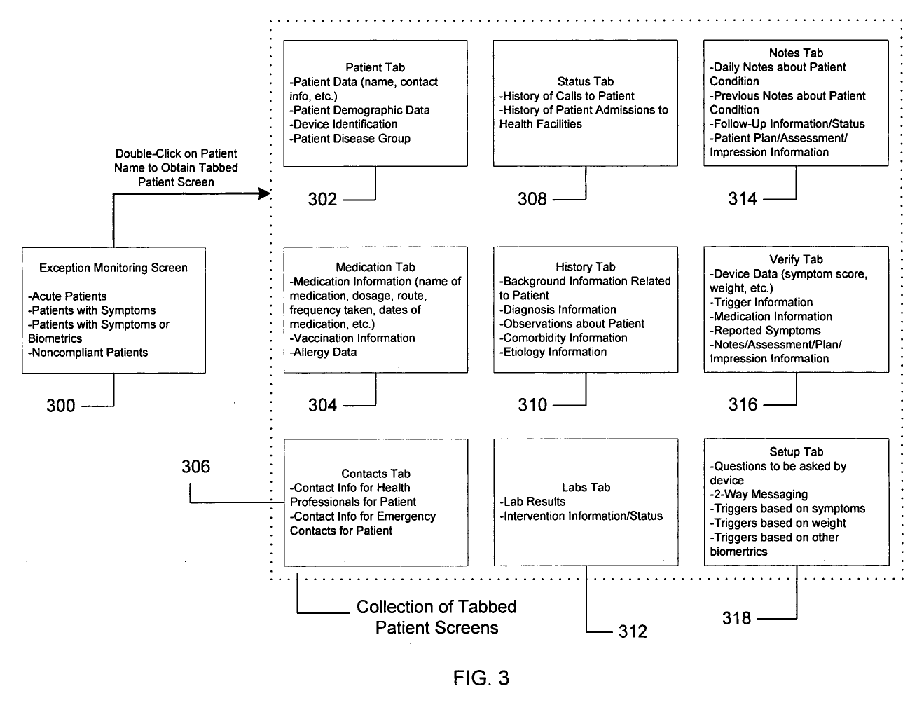

FIG. 3 depicts a schematic view of a user interface in accord with the scheme described with reference to FIGS. 1 and 2. As can be seen from FIG. 3, the user interface includes an exception monitoring screen 300 from which one is able to access a plurality of associated patient screens 302-318. The exception monitoring screen 300 presents information about a sub-populace of users 112. Specifically, the exception monitoring screen 300 presents information including: (1) the identity of users who have been identified as having an acute condition; (2) the identity of users whose answers to questions indicate that they need attention by a health care professional; (3) the identity of users whose biometric data and/or answers to questions indicate that they need attention by a health care professional; (4) and the identity of users who failed to user their device in the last day(s).

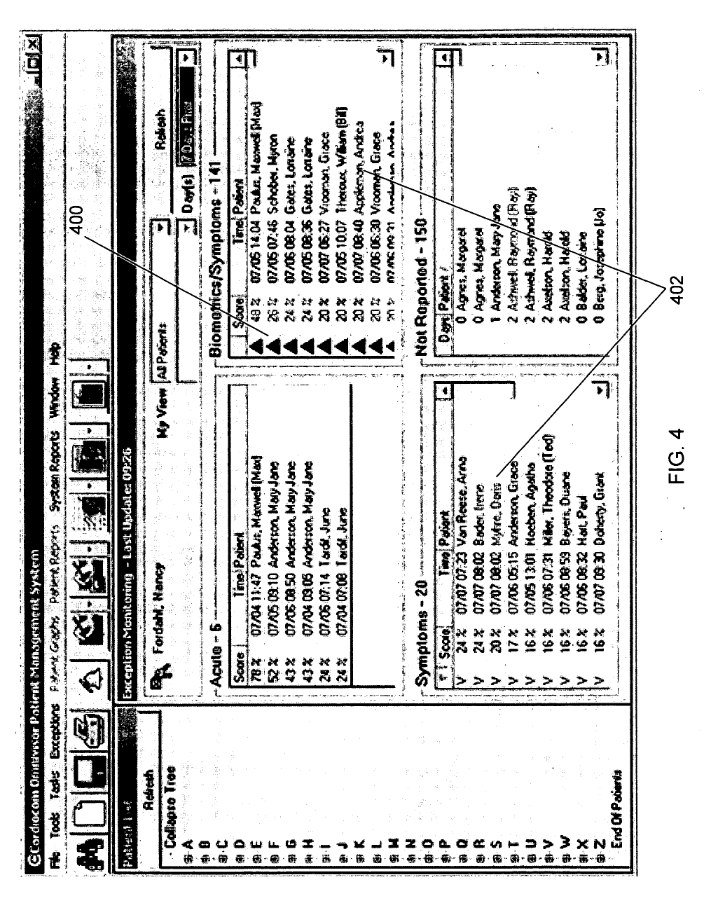

FIG. 4 depicts an example of an exception monitoring screen 300. Of particular note therein are the color-coded icons 400. The icons 400 appear next to user names whose biometric data and/or answers to questions indicate that they need attention by a health care professional. The color of the icon indicates the reason the reason for which the user name has been added to the list. For example, a yellow icon may indicate that the user 112 was below his or her minimum weight. Similarly, a blue icon may indicate that the user 112 is above his or her maximum allowed weight plus trigger value. Finally, a magenta icon may indicate that a user 112 gained or lost more than a given number of pounds in a given number of days. This topic is discussed in greater detail, below. Additionally, some user names (identified by reference numeral 402) are presented in a color different from the remainder of the text on the screen (e.g., presented in green, while the remainder of the text is black). Such a color designation indicates that an attempt has already been made to contact the particular user 112. This is discussed in greater detail, below.

Returning to FIG. 3, a plurality of associated patient screens 302-318 are depicted as being accessible from the patient monitoring screen 300. The associated patient screens present 302-318 information related to a particular user. The screens may be associated in any manner, ideally being associated so that any of the screens may be accessed with a single mouse click while viewing any other associated screen. One way in which this goal may be accomplished is to define each of the screens 302-318 as separate tabs of a single window. Thus, double-clicking a user name presented on the exception monitoring screen 300 results in opening of a patient window populated with information concerning the user whose name was double-clicked. The patient window is presented with tabs, so that selection of one of the tabs results in viewing of a screen associated with the tab.

As shown in FIG. 3, the patient window may have nine tabs, although in principle a patient window may be composed of a greater or lesser number of tabs. The nine tabs depicted in FIG. 3 are: (1) the patient tab 302; (2) the medication tab 304; (3) the contacts tab 306; (4) the status tab 308; (5) the history tab 310; (6) the labs tab 312; (7) the notes tab 314; (8) the verify tab 316; and (9) the setup tab 318.

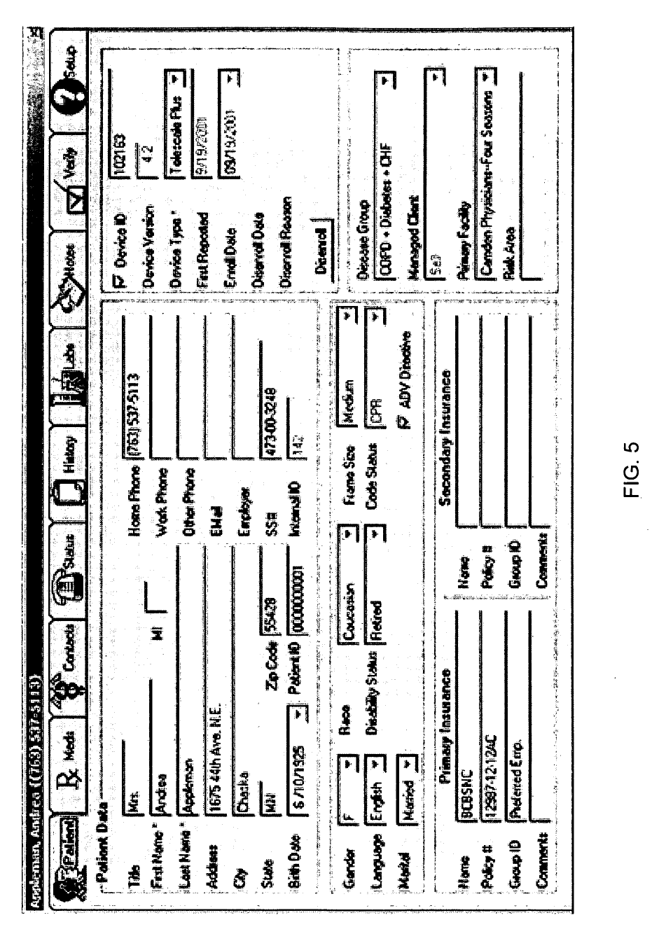

The screen associated with the patient tab typically presents user data (patient name, address, etc.), user demographic data, device information, and information related to the disease group to be monitored. An example of a screen associated with the patient tab is depicted in FIG. 5.

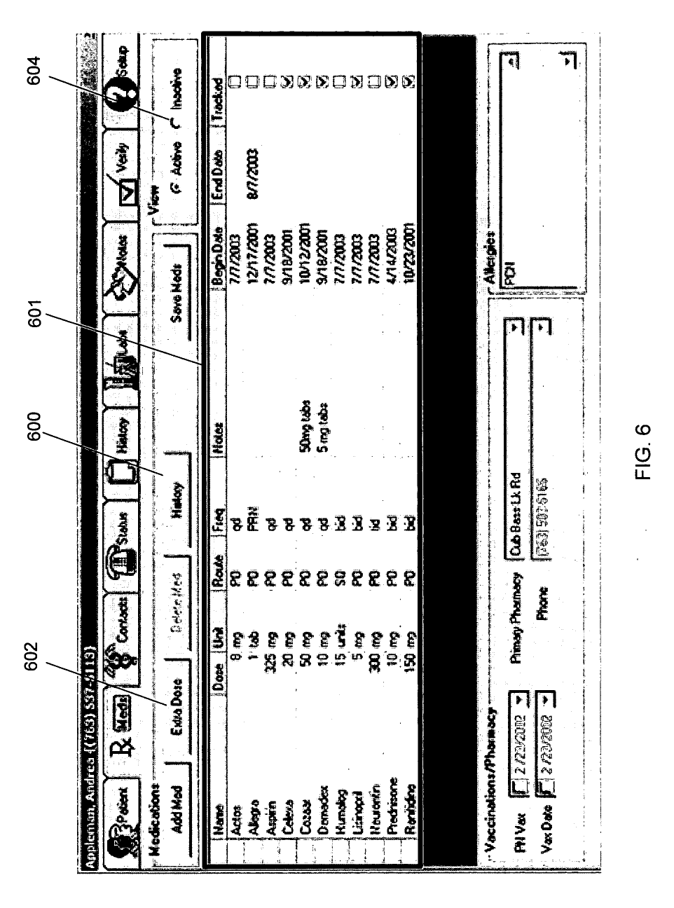

The screen associated with the medication tab typically presents medication information pertaining to the user 112 (name of medication, dosage, route of intake of medication, frequency with which the medication is taken, and dates during which the medication is taken). The system also stores and displays the history of all medications, and their associated dose, route of intake, frequency, notes, date on which the user began taking the medication and date on which the user 112 stopped taking the medication. In other words, the system allows for accesss to all medication information in the past. This information may be obtained by selection of a particular medication and selection of the history button 600. Thus, for example, if the dosage of Allegra was changed from two tablets to one tablet at some point in the past, a screen is opened showing the dates upon which Allegra was taken at a dosage of one tablet per day, and the date at which the dosage changed to two tablets per day. The screen of FIG. 6 also contains an extra dose button 602. This button 602 permits an extra dose of a particular medication to be perscribed for a defined period. For example, if a second tablet of Allegra were to be perscribed for a period extending from a first date to a second date, then a second entry for Allegra is presented in the medications field 601 on the screen of FIG. 6. The second entry indicates that a single tablet is to be taken (the first entry indicating a dosage of one tablet is also present, meaning that a total of two tablets are to be taken by the user 112) for the dates beginning on the first date and ending on the second date. The second entry may be highlighted in order to draw attention to the field. After the second date has elapsed, the second entry is removed from the medications field of the screen presented on FIG. 6. The screen of FIG. 6 may also contain an inactive button 604. Selection of the inactive button causes the medication field 601 of FIG. 6 to be populated with information concerning medications the user 112 had taken at one point in time, but is no longer taking.

The screen of FIG. 6 may also present vaccination information and allergy information. In short, the screen presents information sufficient to inform a health care professional about which medications the user 112 may be using or may have used in the past. An example of a screen associated with the medication tab is depicted in FIG. 6.

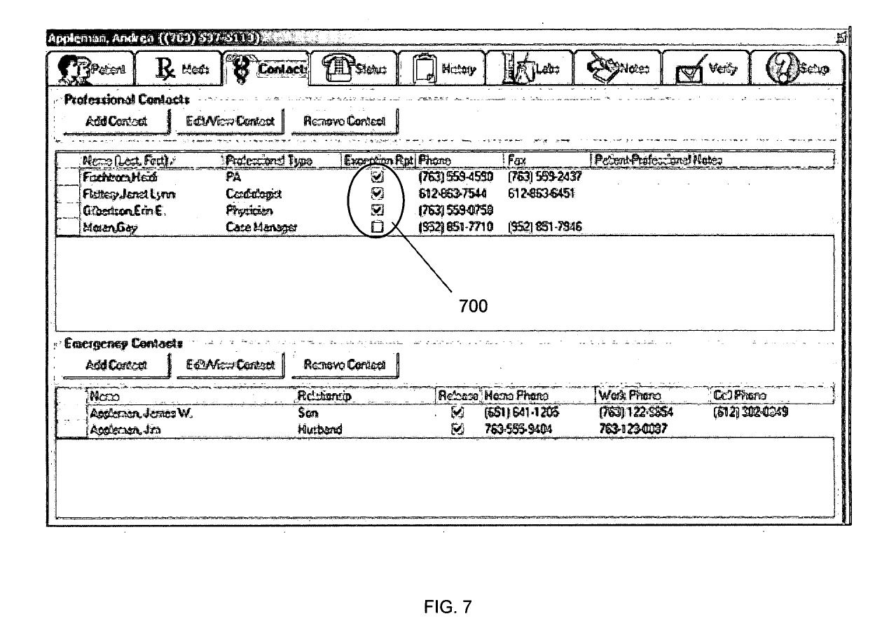

The screen associated with the contacts tab typically presents the names and contact information of the health care professionals that care for the user 112. Also, the screen typically presents the name and contact information of individuals to be contacted in case of an emergency involving the user 112. An example of a screen associated with the contacts tab is depicted in FIG. 7. Of particular note therein are checkboxes 700 by which a user may indicate that a particular health care professional be contacted in the event that an exception is declared with respect the particular user 112. Although the contact screen depicted in FIG. 7 shows data fields for presenting contact information such as telephone numbers for voice and fax, other data fields maybe present. For example, the datastore 106 may store e-mail addresses or other electronic contact information, such as a pager number, for each health care professional listed on the screen. Such additional contact data may or may not be presented on the screen. Further, although not depicted, the screen may contain a field that designates which health care professionals care for the user 112 on weekends or other designated days of the week. For example, a checkbox labeled “weekend” may be provided next to the name of each health care professional listed on the screen. A check in the check box indicates that the particular health care professional cares for patients on the weekend. The system may operate on the assumption that a health care professional does not provide services on the weekend if no check is present in the checkbox.

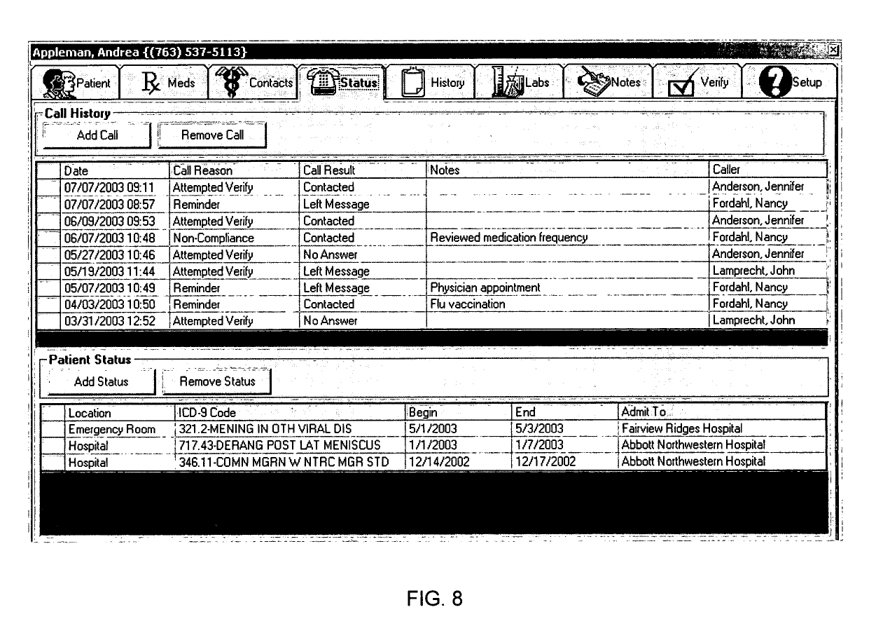

The screen associated with the status tab typically presents a history of calls placed from the call center to the patient. The history may include data fields presenting information relating to the date of the call, the reason for the call, the result of the call (no answer, spoke with the user, left a message, etc.), notes relating to the call, and the name of the party who placed the call. Additionally, the screen may present data related to any hospitalization of the patient. An example of a screen associated with the status tab is depicted in FIG. 8.

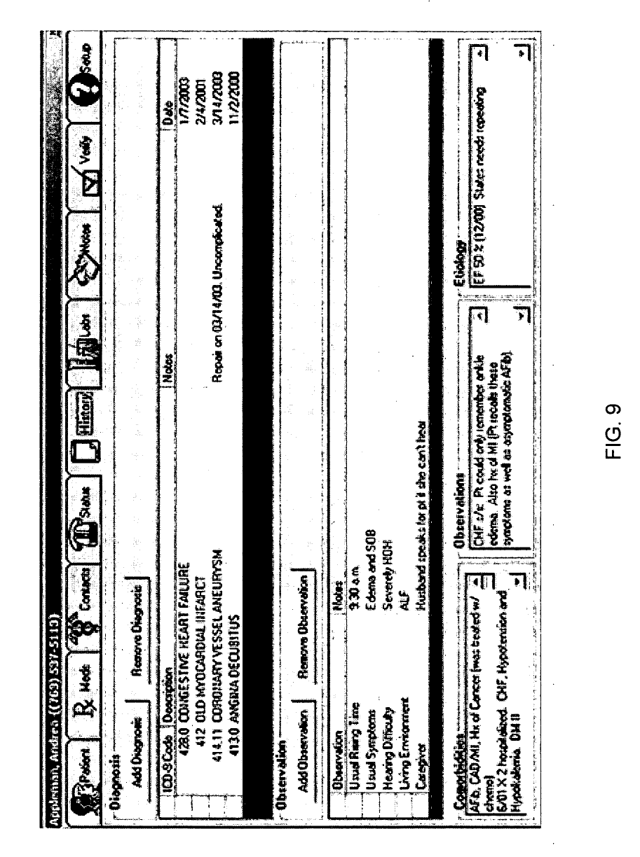

The screen associated with the history tab typically presents background information related to the user 112. Such information includes: diagnosis information, observations about the patient, comorbidity information, and etiology information. An example of a screen associated with the history tab is depicted in FIG. 9.

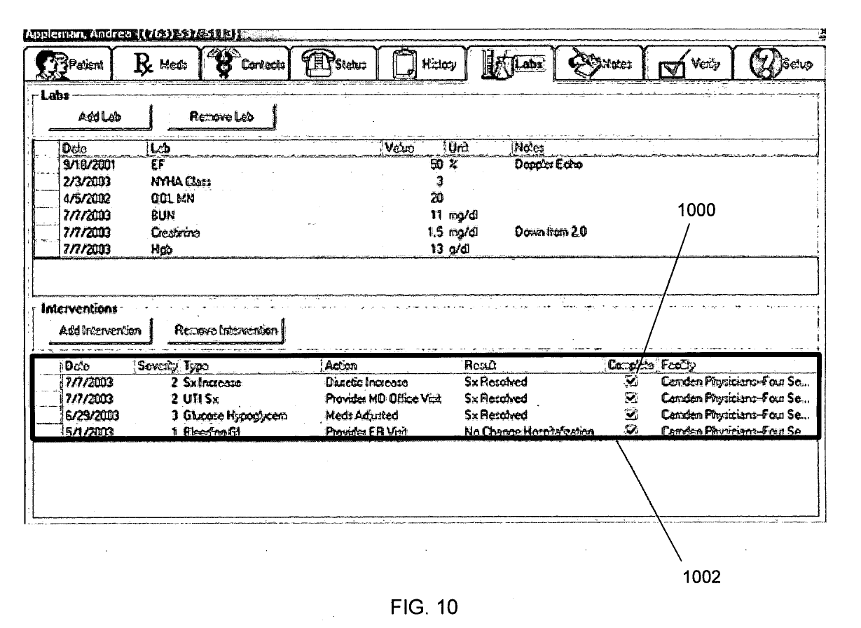

The screen associated with the labs tab typically presents lab results. The screen also typically presents a list of interventions, including the date of the intervention, an indication of the condition to be intervened, an indication of the severity of the condition, an indication of the intervention action, the result observed, an indication of whether the intervention is complete (i.e., an indication of whether a sufficient duration has elapsed to observe the efficacy of the intervention action-this indication is identified by reference numeral 1000), and the facility undertaking the intervention action. More discussion related to the presentation and creation of intervention data is presented below. An example of a screen associated with the labs tab is presented in FIG. 10.

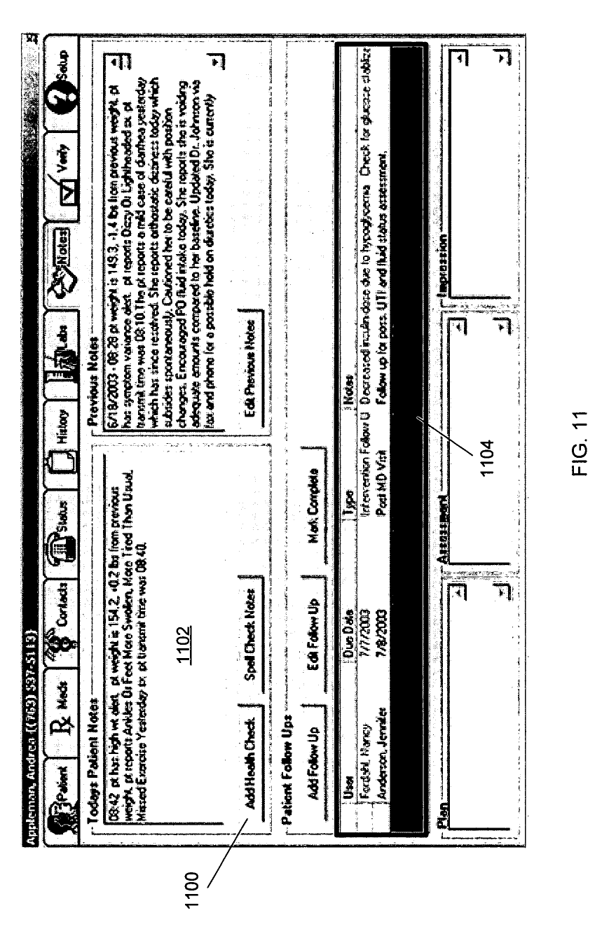

The screen associated with the notes tab typically presents a data field in which to enter daily notes about the user's 112 condition and a data field in which to view previously entered notes concerning the user's 112 condition. The screen also typically includes data fields 1104 in which to view and schedule follow-up contacts with the user 112. The screen typically possesses a button or other means by which a follow-up entry may be identified as having been completed. Finally, the screen may present fields in which to enter plan information, assessment information, and impression data. An example of a screen associated with the notes tab is depicted in FIG. 11. Notably, the screen contains a button 1100 labeled “Add Health Check.” Selection of this button causes the system to automatically enter notes into the daily notes field 1102, based upon the answers provided by the user 112 during his or her interaction with the device 110 and based upon the biometric data obtained by the device 110 during the user's 112 interaction therewith. This feature is discussed in greater detail, below.

The screen associated with the verify tab typically presents data collected from the device 110, symptoms reported by the user 112 during his or her last interaction with the device 110, and at least some of the trigger conditions related to the biometric data collected by the device 110. Further, the screen may present medication information displayed/entered from the screen associated with the medication tab. Still further, the screen may display the notes, assessment information, plan information, and impression data displayed/entered from the screen associated with the status screen.

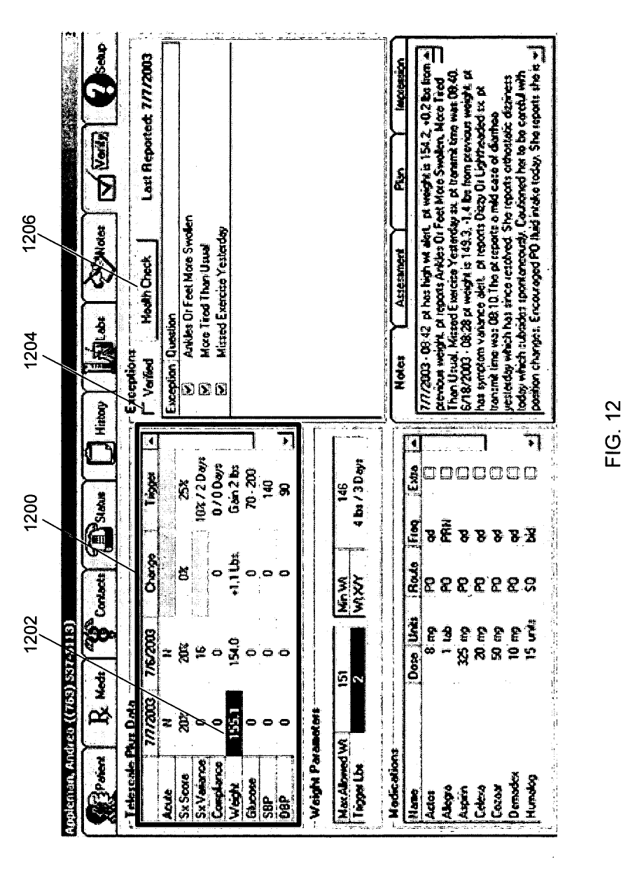

An example of a screen associated with the verify tab is depicted in FIG. 12. The screen has a portion 1200 that displays data collected by the device 110. The device data portion 1200 presents data arranged in rows and columns. Each row contains data related to a value (e.g., weight, symptom score, etc.) that has a trigger condition associated with it. If the value satisfies the trigger condition, an exception is declared for the user 112. If the value fails to satisfy the trigger condition, no exception is declared. If a particular value satisfies a trigger condition, the cell in which the value is presented is highlighted with a color. For example, cell 1202 is highlighted, indicating that the current weight of the patient is causing an exception. This feature immediately draws the attention of the operator to the particular measurements causing concern. This is discussed in greater detail, below. Information relating to setting of the trigger conditions is discussed below. Also of note on this screen is a “verified” checkbox 1204. By placing a check in this checkbox 1204, the operator indicates that the user's exception has been verified by a phone call. Once this checkbox 1204 is selected, the user's name is removed from the appropriate list on the exception monitoring screen 300. Also of note on this screen is the “health check” button 1206. Selection of this button 1206 causes opening of a window that permits the operator to change one or more of the answers provided by the user 112 during the user's interaction with the device 112. Although not depicted on this screen, this screen may also contain a button that provides access to a expert system that helps an operator ask a set of questions to specify a diagnosis and recommend a treatment for a health care professional to review. This is discussed in greater detail below.

The screen associated with the setup tab typically presents data such as questions set to be activated by the device, textual messages to be transmitted to the device via a two-way messaging feature, and trigger conditions based on the user's 112 answers to questions during his interaction with the device 110, trigger conditions based upon measured weight data, and trigger conditions based upon other biometric data.

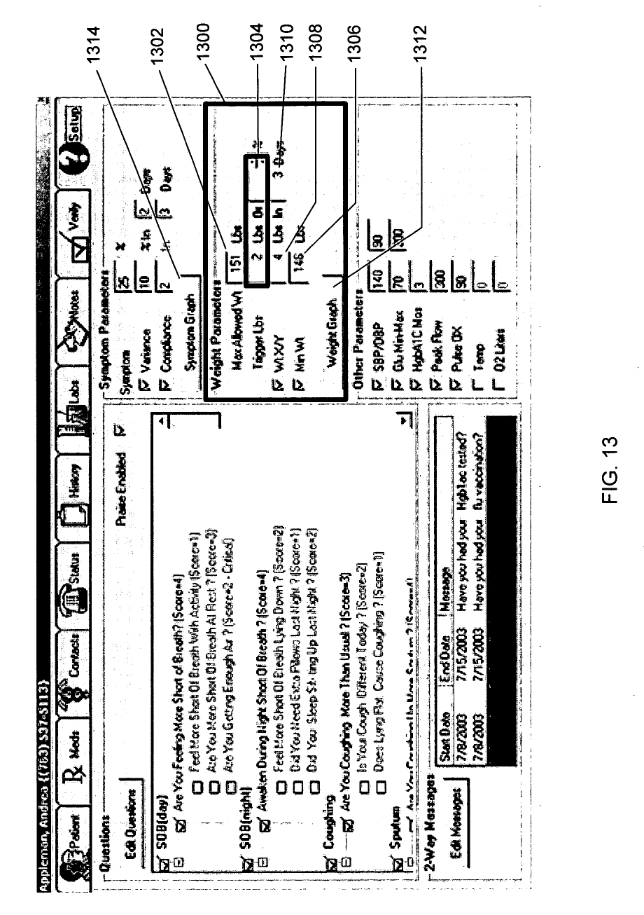

An example of a screen associated with the setup tab is depicted in FIG. 13. Of note therein is a portion 1300 of the screen devoted to setting of trigger conditions based upon measured weight data. The portion permits the operator to specify three types of trigger conditions that may be satisfied by the user's weight: (1) a trigger condition satisfied if the user's weight is greater than the maximum allowed weight 1302 plus the trigger weight 1304 (expressed in lbs or as a percentage of the maximum allowed weight 1302); (2) a trigger condition satisfied if the user's weight is less than the minimum allowed weight 1306; and (3) a trigger condition satisfied if the user 112 gains or loses more than a selected number of pounds 1308 in a selected number of days 1310. Notably, the screen contains a button 1312 entitled “weight graph.” Selection of this button causes a window to open. The window permits the operator to visually compare contemplated trigger settings against historical user weight data, so that the operator can see how frequently an exception would be declared for a given user 112 if the contemplated trigger condition were set by the operator. This is discussed in greater detail below.

Clinical Note Generator

As alluded to above with reference to FIG. 11, selection of a button 1100 labeled “add health check” automatically causes notes to be entered into the daily notes field 1102. The notes are based upon the answers provided by the user 112 during his or her interaction with the device 110, and based upon the biometric data obtained by the device 110 during the user's 112 interaction therewith.

The workstations 102 may be programmed with a set of conditions against which the user input (i.e., the user's answers provided during his or her interaction with the device 110) is compared. For each answer, an appropriate condition is retrieved, and the answer is compared against a condition. If the condition is not satisfied, no text is generated at all. If, on the other hand, the condition is satisfied, the answer is processed by a text generating unit. The text generating unit is programmed with a set of rules that matches the particular answer to a textual phrase that is entered into the daily notes field.

For example, assume the user 112 answered “yes” to the question “are your ankles and feet swollen today?” Initially, the answer is compared against a condition retrieved from the aforementioned condition set. Thus, for example, the retrieved condition requires that the user answer “yes” for any text to be generated at all. If the user had answered “no”, no text is generated. This prevents the daily notes field 1102 from being populated with a mass of textual notes that record that fact that a patient was not experiencing a symptom. Since the user answered “yes”, the answer is processed by the text generating unit. The text generating unit matches the answer to a text string that reads “pt experiencing swollen ankles and feet.” This text string is entered into the daily notes field 1102. This procedure is repeated for each answer provided by the user and for each biometric value obtained by the device. In the case of a biometric measurement, the value of the measurement may be inserted into the text string obtained by the text generating unit (e.g., “pt weight is 178 pounds”, where “178” is inserted into the text string by the text generating unit.)

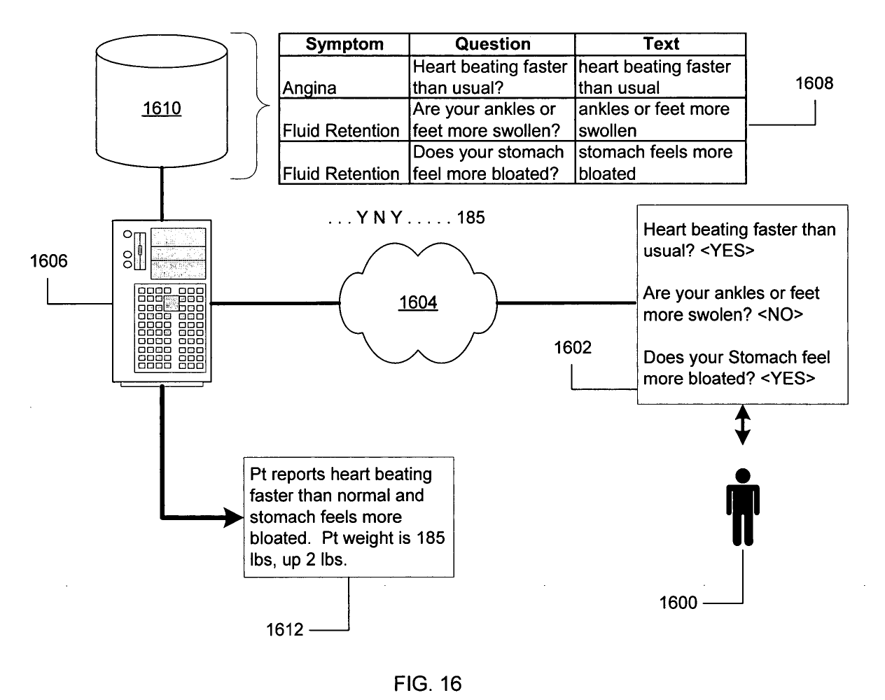

FIG. 16 depicts an embodiment of the above-described process. In FIG. 16, a user 1600 is depicted as interacting with a device 1602 that is posing a series of questions. Three of the questions are presented for the sake of illustration. The questions are: (1) Heart beating faster than usual? (2) Are your ankles or feet more swollen? and (3) Does your stomach feel more bloated? As shown in FIG. 16, the user 1600 answers in the affirmative to the first and third question, and in the negative to the second question. The data acquired by the device 1602 is transmitted from the device 1602, across a network 1604, and to a server 1606. A text generation function creates a clinical note 1612 reading: “Pt reports heart beating faster than normal and stomach feels more bloated. Pt weight is 185 lbs, up 2 lbs.”

Per this embodiment, the data arrives at the server 1606 in one-byte units, with each one-byte unit representing a single user answer or single biometric measurement. Each one-byte answer may be associated with its corresponding question by virtue its place within the data set. In other words, the first byte in the data set represents the answer to the first question, the second byte represents the answer to the second question, and so on.

A text generation function running on the server 1606 or running on the workstations 102 uses the sequence number of the answer to index into a table 1608 stored in a datastore 1610. In other words, when accessing the table 1608, the text generation function accesses the first row of the table 1608 when processing the first byte in the data set. Similarly, the text generation function accesses the second row of the table 1608 when processing the second byte in the data set, and so on. The text generation function accesses the table 1608 in order to determine the symptom type corresponding to the answer. For example, the symptom type corresponding to the first answer is “Angina,” while the symptom type corresponding to the third answer is “Fluid Retention.” The text generation function also looks up corresponding clinical text from the table 1608. For example, the text generation extracts the clinical text “heart beating faster than usual” for the first answer. The clinical text “stomach feels more bloated” is extracted for the third answer. Based upon the symptom types, the text generation function employs grammatical rules to construct clinical notes from the clinical text. For example, the text generation function combines “heart beating faster than usual” and “stomach feels more bloated” by affixing the phrase “Pt reports” prior to recitation of the first clinical text phrase, and interposing the term “and” in between the two clinical text phrases to arrive at the clinical note “Pt reports heart beating faster than normal and stomach feels more bloated.”

The text generation function can also create text for biometric measurements. For example, as shown in FIG. 16, the user's 1600 weight is the final byte in the data set transmitted to the server 1606. Based on its location in the data set, the server 1606 is able to identify “185” (which is expressed as 0×B9 in hexadecimal notation) as representing the user's 1600 weight. The text generation function employs rules to combine static text with text chosen based upon the outcome of a comparison to arrive at a text string to be entered in the clinical note. For example, because the user's weight is 185 lbs, the text generation function makes use of static text to create a first clause: “Pt weight is 185 lbs.” The second clause is constructed based upon a comparison of the presently reported weight with the last reported weight. Given the example shown in FIG. 16, the second clause reads “up 2 lbs.” The word “up” is chosen based upon the comparison (the present weight is greater than the last recorded weight). The phrase “2 lbs.” is inserted as the result of a calculation-the difference between the present weight and the last recorded weight is two pounds.

Automatic Contacting of Health Care Professionals in Response to Exception Declaration

As mentioned with reference to FIG. 7, the screen associated with the contacts tab contains contact information by which health care professionals caring for the user 112 may be reached. Further, each health care professional may be designated (by a checkbox 700) as being an individual who should or should not be contacted when the user 112 is declared as having an exception. The workstations 102 may be programmed to take advantage of these data fields in order to automatically contact the proper health care professionals in response to an exception having been declared for one of the users 112.

The following procedure may be executed either immediately following the execution of operation 208 (FIG. 2) or after an exception has been verified by selection of checkbox 1204 (FIG. 12). For each patient for which an exception has been declared, the workstation 102 or server 104 identifies each of the health care professionals to be contacted by making use of the designations presented by the checkboxes 700. For each health care professional to be contacted, contact information, such as an e-mail address, a fax number, or a pager number is retrieved from the datastore 106. Next, the health care professional is contacted automatically by making use of the contact information. For example, the workstation 102 or server 104 may send the contact information to an e-mail service accessible by the machine, so that an e-mail is sent to the health care professional. The e-mail alerts the health care professional to the fact that his or her patient has been identified as being in need of medical assistance. Optionally, the workstation 102 or server 104 may retrieve from the datastore 106 some or all of the data on the screen associated with the verify tab, so that it may be inserted into the text of the e-mail. This allows the health care professional to make a preliminary evaluation. Alternatively, the health care professional may be paged or faxed. The page or fax may also optionally contain some or all of the data on the screen associated with the verify tab, so that the health care professional is able to make a preliminary evaluation. The body of the communication (page, fax, e-mail, etc. may be composed making use of the clinical note generator described herein).

Optionally, the workstations 102 may be programmed to take advantage of a designation field (such as a checkbox) that indicates whether or not a particular health care professional is to be contacted on weekends. Thus, for example, if the exception is generated on a Saturday or Sunday, health care professionals having a “weekend” checkbox marked are contacted.

Automatic Creation of Intervention

As mentioned with reference to FIG. 10, the screen associated with the labs tab presents intervention data. An intervention is a proposed treatment to counteract a symptom experienced by the user 112. Each time an intervention is undertaken, an entry is created in the intervention data field 1002. Each entry may contain the date the intervention was undertaken, an indication of the condition to be counteracted, an indication of the severity of the condition, an indication of the intervention action, the result observed, an indication of whether the intervention is complete (i.e., an indication of whether a sufficient duration has elapsed to observe the efficacy of the intervention action—this indication is identified by reference numeral 1000), and the facility undertaking the intervention action. The workstation 102 may be programmed to automatically create an entry in the intervention data field 1002 for each exception that is declared for a particular user 112.

The following procedure may be executed either immediately following the execution of operation 208 (FIG. 2) or after an exception has been verified by selection of checkbox 1204 (FIG. 12). For each patient for which an exception has been declared, the workstation 102 may create an entry in the intervention data field, automatically filling in the date, the type, and/or the severity. The severity value may be arrived at by comparing a value that is compared to a trigger condition with the extent to which the value equals or surpasses the trigger condition. For example, if the user's weight is above the maximum allowed weight 1302 (FIG. 13) by more than a particular percent of the maximum allowed weight 1302 (FIG. 13) (e.g., more than 2%), the severity may be assigned a value of “1.” If, however, the user's weight exceeds the maximum allowed weight by an even greater percentage (e.g., more than 5%) of the maximum allowable weight 1302, then the severity may be assigned a value of “2”. Finally, if, the user's weight exceeds the maximum allowed weight by yet an even greater percentage (e.g., more than 10%) of the maximum allowed weight 1302, then the severity may be assigned a value of “3”.

Generation of Reminders

As discussed with reference to FIGS. 10 and 11, the screens associated with the labs tab and the notes tab may contain data fields for presentation/entry of interventions 1002 and follow-ups 1104. As also discussed previously, an entry in the intervention field 1002 contains an indication of whether the intervention is complete. The indication may come in the form of a checkbox, such as checkbox 1000. Similarly, an entry in the follow-up field may contain a due date entry, and may be marked complete by selection of a button 1006 labeled “mark complete”. (Selection of the mark complete button 1006 causes the follow-up entry to disappear from the follow-up data field 1104).

To ensure that interventions and follow-ups are not forgotten, reminder messages may be automatically generated. For example, the workstations 102 may be programmed to identify unresolved interventions and follow-ups at a designated time (such as immediately following power-up of the computer or at a specified time of the day). For each identified intervention and follow-up, an e-mail message identifying the user 112 and the associated intervention or follow-up may be generated and sent to an operator at the call center. Alternatively, a single e-mail may contain a list of all open interventions and/or follow-ups for all users 112. Still alternatively, a window may be automatically opened on the computer. Such a window lists each user with an open intervention or follow-up and identifies the intervention or follow-up.

Trigger Graphs

As described with respect to FIG. 13, the operator may set three trigger conditions based upon the user's 112 measured weight: (1) a trigger condition satisfied if the user's weight is greater than the maximum allowed weight 1302 plus the trigger weight 1304 (expressed in lbs or as a percentage of the maximum allowed weight 1302); (2) a trigger condition satisfied if the user's weight is less than the minimum allowed weight 1306; and (3) a trigger condition satisfied if the user 112 gains or loses more than a selected number of pounds 1308 in a selected number of days 1310. Further, as alluded to earlier, the operator may set a trigger condition based upon a symptom score earned by the user 112 during his interaction with the device 110. (When the user answers a question so as to indicate the presence of a symptom, a score is earned. The value of the score varies based upon the significance of the symptom. After all of the questions have been answered, the scores are summed and a raw symptom score is arrived at. The raw symptom score is divided by the total possible symptom score to arrive at a symptom score expressed as a percentage.) This particular trigger condition may be satisfied when the symptom score expressed as a percentage exceeds a selected threshold.

The process of setting the aforementioned trigger conditions is difficult, due to the number of variables involved. Put simply, the trigger conditions should be set low enough so that an exception is declared when the user 112 should have professional health care attention, but high enough to minimize the occurrence of minimize “false alarms”.

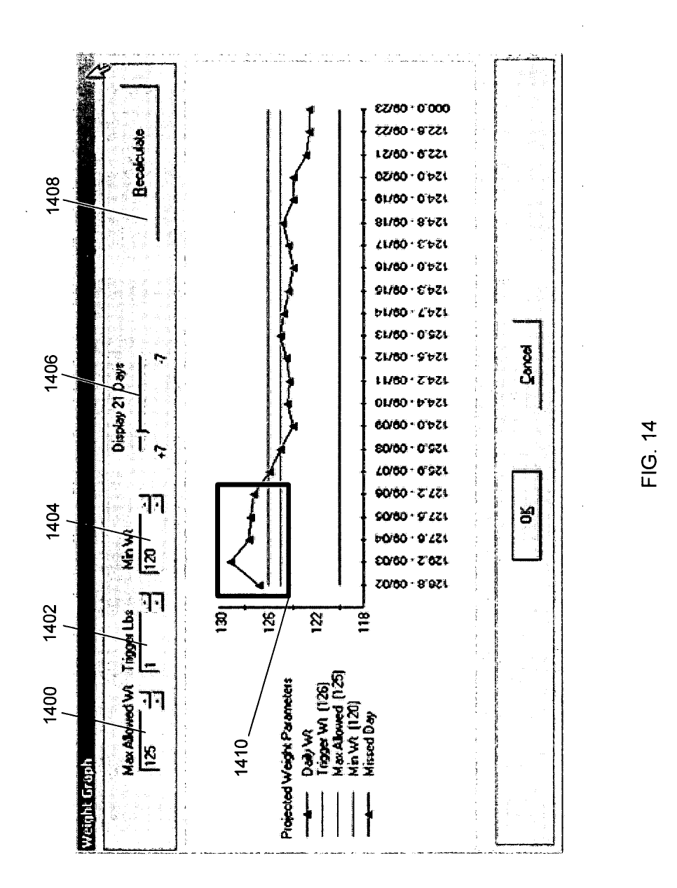

As can be seen from FIG. 13, the screen associated with the setup tab may have buttons 1312 and 1314 labeled “weight graph” and “symptom graph.” Selection of the button 1312 labeled “weight graph” opens a window depicted in FIG. 14. As can be seen in FIG. 14, the weight graph window contains data fields 1400, 1402, and 1404 which permit the user to select proposed trigger settings for maximum allowed weight, trigger pounds, and minimum weight, respectively. A display days slider 1406 and recalculate button 1408 are also included on the window. Selection of the recalculate button causes the workstation perform the following steps. The workstation 102 retrieves from the datastore 106 the weight measurements recorded by the device 110 over a span of days indicated by the display days slider (e.g., per the example shown in FIG. 14, weight measurements for the preceding 21 days are retrieved). Next, the weight measurements are plotted along a graph, having an x axis representing the date on which the measurements were taken, and a y axis representing weight. Also, the minimum weight, as set in data field 1404 is plotted on the graph, as is the maximum allowed weight, as set in data field 1400. Finally, the trigger weight (equal to the sum of the maximum allowed weight and the trigger pounds) is plotted on the graph. Such a graph may be viewed by the operator to determine on which days the proposed trigger setting would have yielded an exception. For example, according to the example shown in FIG. 14, an exception would have been on the days identified by reference numeral 1410. If the outcome is acceptable, the operator may select the OK button, and the proposed settings are transferred to the datastore 106 and used as the real values for the trigger conditions. Otherwise, the operator may select the cancel button, and the window will be closed without having changed the pre-existing trigger condition values.

Although not depicted on FIG. 14, the window may contain data fields permitting the selection of proposed values for the trigger condition satisfied upon the user 112 gains or loses more than a selected number of pounds (represented by X) in a selected number of days (represented by Y). In other words the window may contain data fields for selection of values for X and Y. Per such a scenario, selection of the recalculate button 1408 causes the workstation 102 to perform the following steps. For each weight point plotted on the graph, the workstation 102 looks Y number of days into the past and determines by how many pounds the user's 112 weight has changed. If the weight change exceeds X, a visual indicator is presented for that particular weight point (e.g., the weight point may be presented in a different color). By execution of the preceding steps, the resultant graph permits an operator see the impact of proposed trigger values for X and Y.

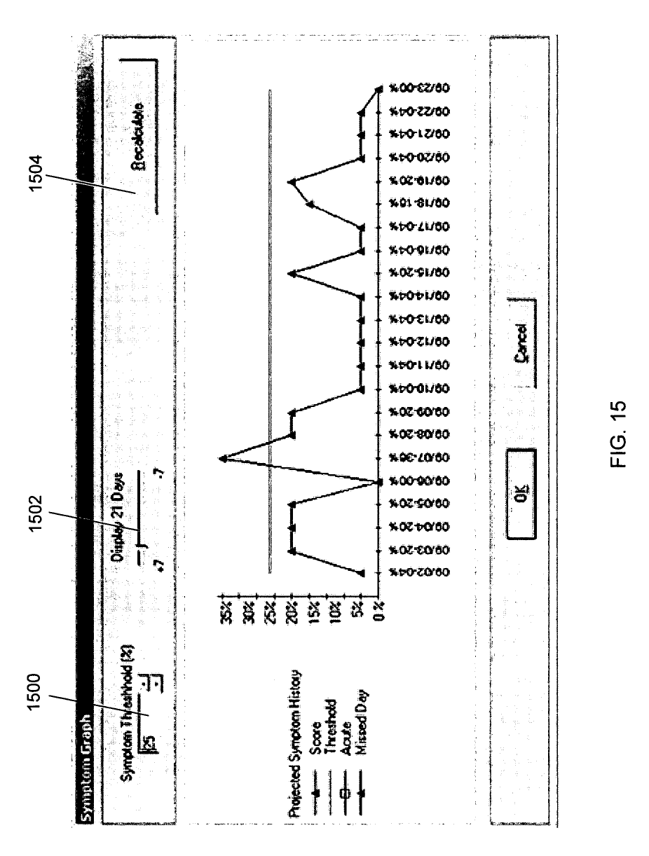

Returning to FIG. 13, selection of the button 1314 labeled “symptom graph” opens a window depicted in FIG. 15. As can be seen in FIG. 15, the symptom graph window contains data field 1500 which permits the user to select a proposed trigger setting for the symptom score threshold. A display days slider 1502 and recalculate button 1504 are also included on the window. Selection of the recalculate button 1504 causes the workstation perform the following steps. The workstation 102 retrieves from the datastore 106 the symptom score values earned by the user 112 over a span of days indicated by the display days slider (e.g., per the example shown in FIG. 15, symptom scores for the preceding 21 days are retrieved). Next, the symptom scores are plotted along a graph, having an x axis representing the date on which the symptom scores were earned, and a y axis representing the symptom score expressed as a percentage. Finally, the proposed symptom score threshold, as set in data field 1500, is plotted on the graph. Once again, an operator may view the graph to determine whether the outcome is acceptable. If the outcome is acceptable, the operator may select the OK button, and the proposed settings are transferred to the datastore 106 and used as the real values for the trigger conditions. Otherwise, the operator may select the cancel button, and the window will be closed without having changed the pre-existing trigger condition values.

Automatic Calling of Noncompliant Users

As discussed with reference to FIG. 3, the exception monitoring screen may present a list of users who failed to interact with their device in the preceding day. Such users may need to be contacted to determine the reason for having failed to use their device.

The workstations 102 may be coupled, either directly or indirectly (such as via the server 104), to a telephone interface unit. At a specified time of day, the telephone interface unit may be supplied with the telephone numbers corresponding to the user names presented on the exception monitoring screen for noncompliance. In response to being supplied with a telephone number, the telephone interface unit calls the supplied number. Upon answering of the telephone, the telephone interface unit plays a pre-recorded message to the party. The pre-recorded message may simply remind the user to interact with his or her device. Alternatively, the message may ask the user to press touch-tone telephone buttons to indicate the reason for the noncompliance. For example, the prerecorded message may say “press one if you have already reported, press two if you have no symptoms and will report again tomorrow, press three if your device is out of order, and press four to speak with a health care professional now.” In response to a selection of a touch-tone button, the telephone interface unit returns a data value indicating which button had been selected by the user, thanks the user, and hangs up. The workstation 102 may simply remove the name of the user from the noncompliant list if the user indicated that he or she has already reported, or if the user indicated that he or she has no symptoms and will report tomorrow. On the other hand, if the user 112 indicates that his or her device is out of order, an e-mail may be generated and transmitted to the operator, instructing the operator to schedule maintenance (or schedule the delivery of a replacement device) for the user's device. Finally, if the user 112 indicates that he or she needs to speak with a health care professional immediately, the call may be routed to a health care professional.

To further personalize the call, the nurse or health care professional assigned to the user 112 may record the message presented to the user during the automatic call back operations. Thus, the recording is in a voice familiar to the user 112.

Color Coding

As mentioned with reference to FIG. 4, the exception monitoring screen contains color-coded icons 400. The icons 400 appear next to user names whose biometric data and/or answers to questions indicate that they should have attention by a health care professional. The color of the icon indicates the reason for which the user name has been added to the list. For example, a yellow icon may indicate that the user 112 was below his or her minimum weight. Similarly, a blue icon may indicate that the user 112 is above his or her maximum allowed weight plus trigger value. Finally, a magenta icon may indicate that a user 112 gained or lost more than a given number of pounds in a given number of days.

For the sake of convenience for the operator, an identical color coding scheme is used on the screen associated with the verify tab. Returning to FIG. 12, one can see that cell 1202 therein is highlighted. The highlighting indicates that the value contained in cell 1202 is the source of an exception. The color of the highlighting may be identical to that of the color of the icon on the exception monitoring screen. In this way, the operator can immediately be alerted to which variable caused the exception, and the reason for the cause of the exception.

Additionally, as mentioned with reference to FIG. 8, the screen associated with the status tab may present a call history. Upon addition of an entry into the call history field, the workstation 102 may perform the following steps. The workstation 102 may determine whether the data field relating to the reason for the call indicates that the call was made in an attempt to verify an exception. If so, the workstation 102 may seek the name of the user 112 to whom the call was placed on any list presented on the exception monitoring screen. Upon finding the name, the workstation 102 may display the name in a particular color (e.g., green) to indicate that the party has been called at least once to attempt a verification.

Expert System

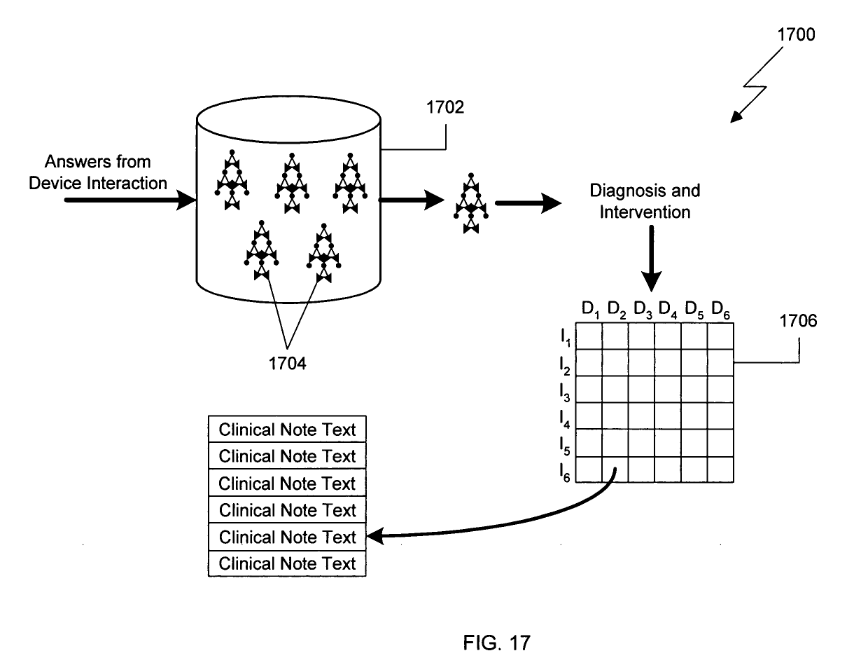

An expert system may be provided to assist the operator during his or her verification call to the user 112. An example of an expert system 1700 is depicted in FIG. 17. The expert system 1700 includes a datastore 1702 that has a plurality of decision trees 1704 programmed within it. A decision tree is a set of questions designed to mimic the questioning process conducted by a health care professional. According to a decision tree structure, the n^{th} question posed to a user is a function of the user's answer to the n−1^{th} question. By extrapolation, per a decision tree structure, the n^{th} question posed to a user is a function of every answer to every question preceding the n^{th} question. Traversal of a decision results in one of two results: (1) a series of questions is posed, until a preliminary diagnosis and intervention is determined; or (2) a series of questions is posed, until the expert system is unable to arrive at a preliminary diagnosis and intervention, and has no further questions to ask.

As depicted in FIG. 17, the expert system retrieves from the datastore 106 the data acquired by the device 110 during the user's last interaction with it. Based on this data, one of a plurality of decision trees 1704 is selected by the expert system.

The expert system 1700 presents the first question from the decision tree to the operator. The operator poses the question to the user 112, and records the user's answer. The structure of the chosen decision tree determines the number of potential questions which can be posed after posing of the first question. For example, the expert system may be designed so that the user 112 may answer in one of a finite number or ways (e.g., the user may be asked a yes-no question, or may be asked to rank the severity of a symptom on a scale of one-to-ten). The decision tree structure associates a second question with each of the finite number of answers to the first question (e.g., if the answer is “yes”, then ask question_{A} as the second question; if the answer is no, then ask questions as the second question). The decision is traversed in the aforementioned pose question-record answer-get new question format, until one of two conditions come about: (1) a preliminary diagnosis and intervention (i.e., treatment) is arrived at; or (2) there are no more questions to ask.

If a preliminary diagnosis and intervention is arrived at, a two-dimensional matrix 1704 may be accessed by the expert system 1700. The two dimensional matrix may be indexed into by a first variable, representing the diagnosis, and a second variable, representing the intervention. By indexing into the array 1704, a pointer may be obtained. The pointer may be used to obtain the first character of a text string that is to be used as a clinical note describing the telephonic interaction with the patient, the preliminary diagnosis, and the preliminary intervention. The clinical note may then be communicated to a health care professional for review.

If, on the other hand, a preliminary diagnosis and intervention are not arrived at by traversal of the decision tree, the set of answers may be communicated to a clinical note generator, such as the clinical note generator described with reference to FIG. 16, to construct a clinical note detailing the user's symptoms. The clinical note may then be communicated to a health care professional for review.

Automatic Optimization of Trigger Conditions

As described above, an operator may set a trigger condition based upon a symptom score earned by the user 112 during his interaction with the device 110. (When the user answers a question so as to indicate the presence of a symptom, a score is earned. The value of the score varies based upon the significance of the symptom. After all of the questions have been answered, the scores are summed and a raw symptom score is arrived at. The raw symptom score is divided by the total possible symptom score to arrive at a symptom score expressed as a percentage.) This particular trigger condition may be satisfied when the symptom score expressed as a percentage exceeds a selected threshold. Selection of a threshold such as this may be automated in one of several way outlined below.

One scheme by which selection of a threshold may be automated is to retrieve each of the symptom scores expressed as a percentage for a span of time. For example, each of the symptom scores expressed as a percentage may be retrieved for the preceding sixty or ninety day period. Then, the mean of the retrieved symptom scores may be found. The threshold may be automatically set as a function of the mean. For example, the threshold may be set to 105% or 110% of the mean.

Another scheme is described below. Initially, a populace of patients monitored for a particular disease state is identified. Then, a variable strongly correlated with patient risk is selected. For example, number of hospitalizations or ejection fraction may be strongly correlated with patient risk. The patient populace is divided into segments (e.g., into thirds) based upon the variable. For example, the populace of patient in the top third with respect to having had the greatest number of hospitalizations is categorized has “high risk.” The populace of patients in the middle third is categorized as “moderate risk,” and the populace in the lowest third is categorized as “low risk.”

To optimize a threshold for a given user 112, the variable used to divided the patient populace into segments is used to place the user 112 into one of the segments. For example, the user is placed into one of the categories based upon his or her number of hospitalizations or based upon his or her ejection fraction. Next, the expert system finds the mean symptom score for the segment in which the user 112 is placed. As in the previous scheme, the threshold may be automatically set as a function of the mean. For example, the threshold may be set to 105% or 110% of the mean.

Another scheme involves identifying dates on which a particular user was hospitalized. Such dates are stored in the datastore 106 for presentation on the screen associated with the status tab (see FIG. 8). The threshold may be automatically set by examining a short period of time immediately preceding a user's hospitalizations. The average symptom score during the periods of time immediately preceding a user's hospitalizations may be found. Again, as in the previous scheme, the threshold may be automatically set as a function of the mean. For example, the threshold may be set to 105% or 110% of the mean.

Various modifications and alterations of this invention will become apparent to those skilled in the art without departing from the scope and spirit of this invention, and it should be understood that this invention is not to be unduly limited to the illustrative embodiments set forth herein. The invention is understood to be defined solely by the claims appended hereto.

1. A system for determining whether a person should have health care professional attention and for providing clinical notes to the caregiver, the system comprising:
a monitoring device having a microprocessor operably coupled to a memory unit, an input device, an output device, and a communication device, the memory unit being programmed with a set of instructions for posing questions to the person via the output device, receiving answers from the person via the input device, and transmitting the answers to a remote computer via the communication device;
the remote computer being programmed to
determine whether the person should have health care professional attention based at least in part upon the answers entered into the input device; and
automatically generate a clinical note based upon the answers transmitted to the remote computer.

2. The system of claim 1, further comprising:
a datastore accessible by the remote computer;
wherein the datastore stores clinical text associated with the questions posed to the person via the monitoring device; and
wherein the remote computer is programmed to generate the clinical note based at least in part upon the clinical text stored in the datastore.

3. The system of claim 2, wherein the datastore also stores a symptom identifier associated with each of the questions posed to the person via the monitoring device; and
wherein the remote computer is programmed to select a grammatical rule for construction of the clinical note based upon the symptom identifier.

4. The system of claim 1, wherein the clinical note comprises verbiage presenting symptoms reported by the person via the input device.

5. The system of claim 1, wherein:
the monitoring device further comprises a biometric measuring unit operably coupled to the microprocessor;
the memory unit in the monitoring device is further programmed with a set of instructions to cause the biometric measuring unit to take a measurement of the patient, and to transmit the measurement to the remote computer; and
the remote computer is further programmed to generate a clinical note based upon the measurement transmitted to the remote computer.

6. The system of claim 1, wherein the remote computer is further programmed to present a user interface that permits viewing of the clinical note and also permits viewing of a populace of persons identified as potentially needing attention by a health care professional.

7. The system of claim 1, wherein the clinical note is communicated to a health care professional.

8. The system of claim 7, wherein the communication occurs via e-mail.

9. The system of claim 1, wherein the remote computer is further programmed to present questions to be posed to the person using the monitoring device, the questions being used to verify the determination that the person should have health care professional attention.

10. The system of claim 1, wherein the remote computer is further programmed to provide a user interface permitting selection of a disease state for monitoring by the monitoring device.

11. A computer system for interfacing with a monitoring device that poses questions regarding disease state symptoms to a person, receives answers from the person, and transmits the answers to the computer system, the computer system comprising:
a microprocessor operably coupled to a memory unit, an input device, an output device, and a communication device;
wherein the memory unit is programmed with a set of instructions for
determining whether the person should have health care professional attention based at least in part upon the answers transmitted to the computer system; and
generating a clinical note based upon the answers transmitted to the computer system.

12. The computer system of claim 11, further comprising:
a datastore accessible by the computer system;
wherein the datastore stores clinical text associated with the questions posed to the person via the monitoring device; and
wherein the computer system is programmed to generate the clinical note based at least in part upon the clinical text stored in the datastore.

13. The computer system of claim 12, wherein the datastore also stores a symptom identifier associated with each of the questions posed to the person via the monitoring device; and
wherein the remote computer is programmed to select a grammatical rule for construction of the clinical note based upon the symptom identifier.

14. The computer system of claim 11, wherein the clinical note comprises verbiage presenting symptoms reported by the person via the monitoring device.

15. The computer system of claim 11, wherein:
the computer system is further programmed to generate a clinical note based upon a biometric measurement transmitted to the computer system from the monitoring device.

16. The computer system of claim 11, wherein the computer system is further programmed to present a user interface that permits viewing of the clinical note and also permits viewing of a populace of persons identified as potentially needing attention by a health care professional.

17. The computer system of claim 11, wherein the clinical note is communicated to a health care professional.

18. The computer system of claim 17, wherein the communication occurs via e-mail.

19. The computer system of claim 11, wherein the computer system is further programmed to present questions to be posed to the person using the monitoring device, the questions being used to verify the determination that the person should have health care professional attention.

20. The computer system of claim 11, wherein the computer system is further programmed to provide a user interface permitting selection of a disease state for monitoring by the monitoring device.

21. A method, carried out by a computer system, of interfacing with a monitoring device that poses questions regarding disease state symptoms to a person, receives answers from the person, and transmits the answers to the computer system, the method comprising:
determining whether the person should have health care professional attention based at least in part upon the answers transmitted to the computer system; and
generating a clinical note based upon the answers transmitted to the computer system.

22. The method of claim 21, further comprising:
storing, in a datastore, clinical text associated with the questions posed to the person via the monitoring device; and
generating the clinical note based at least in part upon the clinical text stored in the datastore.

23. The method of claim 22, further comprising:
storing, in the datastore, symptom identifiers associated with each of the questions posed to the person via the monitoring device; and
selecting a grammatical rule for construction of the clinical note based upon the symptom identifiers.

24. The method of claim 21, wherein the clinical note comprises verbiage presenting symptoms reported by the person via the monitoring device.

25. The method of claim 21, further comprising:
generating a clinical note based upon a biometric measurement transmitted to the computer system from the monitoring device.

26. The method of claim 21, further comprising:
presenting a user interface that permits viewing of the clinical note and also permits viewing of a populace of persons identified as potentially needing attention by a health care professional.

27. The method of claim 21, further comprising communicating the clinical note to the health care professional.

28. The method of claim 27, wherein the communication occurs via e-mail.

29. The method of claim 21, further comprising:
presenting questions to be posed to the person using the monitoring device, wherein the questions are used to verify the determination that the person should have health care professional attention.

30. The method of claim 21, further comprising:
providing a user interface permitting selection of a disease state for monitoring by the monitoring device.

31. A system for determining whether a person should have health care professional attention, the system comprising:
a monitoring device having a microprocessor operably coupled to a memory unit, an input device, an output device, and a communication device, the memory unit being programmed with a set of instructions for posing questions to the person via the output device, receiving answers from the person via the input device, and transmitting the answers to a remote computer via the communication device;
the remote computer being programmed to
determine whether the person should have health care professional attention based at least in part upon the answers entered into the input device; and
permit entry, storage, and presentation of intervention data.

32. The system of claim 31, wherein the intervention data includes data regarding a symptom to be counteracted and an action to be undertaken to counteract the symptom.

33. The system of claim 32, wherein the intervention data further includes the date upon which the intervention data was entered into the remote computer system.

34. The system of claim 33, wherein the intervention data further includes an indication of whether or not the action has counteracted the symptom.

35. The system of claim 31, wherein the remote computer is further programmed to present a user interface that permits viewing of a populace of persons identified as potentially needing attention by a health care professional.

36. The system of claim 31, wherein the remote computer system is further programmed to present an operator with a set of questions, so that the operator may pose the questions to the person using the monitoring device, in response to the person having been identified as potentially needing attention by a health care professional;
wherein the set of questions are designed to permit a conclusion to be drawn regarding a diagnosis of a symptom reported by the person using the device; and
wherein the set of questions are designed to permit a conclusion to be drawn regarding selection of an intervention appropriate for the diagnosis.

37. The system of claim 36, wherein the remote computer is further programmed to arrive at a preliminary diagnosis and preliminary intervention as a function of the person's answers to the questions posed by the operator.

38. The system of claim 37, wherein the remote computer is further programmed to generate a clinical note based upon the preliminary diagnosis and the preliminary intervention.

39. The system of claim 36, wherein the set of questions is chosen based upon the answers transmitted to the remote computer by the monitoring device.

40. The system of claim 36, wherein:
the monitoring device further comprises a biometric measuring unit operably coupled to the microprocessor;
the memory unit in the monitoring device is further programmed with a set of instructions to cause the biometric measuring unit to take a measurement of the patient, and to transmit the measurement to the remote computer; and
the remote computer is further programmed to choose the set of questions based upon the answers transmitted to the remote computer and the measurement taken by the biometric measurement unit.

41. The system of claim 31, wherein intervention data is automatically entered into the remote computer, in response to the remote computer determining that the person should have health care professional attention.

42. A computer system for interfacing with a monitoring device that poses questions regarding disease state symptoms to a person, receives answers from the person, and transmits the answers to the computer system, the computer system comprising:
a microprocessor operably coupled to a memory unit, an input device, an output device, and a communication device;
wherein the memory unit is programmed with a set of instructions for determining whether the person should have health care professional
attention based at least in part upon the answers entered into the input device; and
permitting entry, storage, and presentation of intervention data.

43. The computer system of claim 42, wherein the intervention data includes data regarding a symptom to be counteracted and an action to be undertaken to counteract the symptom.

44. The computer system of claim 43, wherein the intervention data further includes the date upon which the intervention data was entered into the remote computer system.

45. The computer system of claim 44, wherein the intervention data further includes an indication of whether or not the action has counteracted the symptom.

46. The computer system of claim 42, wherein the computer system is further programmed to present a user interface that permits viewing of a populace of persons identified as potentially needing attention by a health care professional.

47. The computer system of claim 42, wherein the computer system is further programmed to present an operator with a set of questions, so that the operator may pose the questions to the person using the monitoring device, in response to the person having been identified as potentially needing attention by a health care professional;
wherein the set of questions are designed to permit a conclusion to be drawn regarding a diagnosis of a symptom reported by the person using the device; and
wherein the set of questions are designed to permit a conclusion to be drawn regarding selection of an intervention appropriate for the diagnosis.

48. The computer system of claim 47, wherein the computer system is further programmed to arrive at a preliminary diagnosis and preliminary intervention as a function of the person's answers to the questions posed by the operator.

49. The computer system of claim 48, wherein the computer system is further programmed to generate a clinical note based upon the preliminary diagnosis and the preliminary intervention.

50. The computer system of claim 47, wherein the set of questions is chosen based upon the answers transmitted to the remote computer by the monitoring device.

51. The computer system of claim 47, wherein:
the computer system is further programmed to choose the set of questions based upon the answers transmitted to the computer system and a measurement taken by a biometric measurement unit associated with the monitoring device.

52. The computer system of claim 42, wherein intervention data is automatically entered into the computer system, in response to the computer system determining that the person should have health care professional attention.

53. A method, carried out by a computer system, of interfacing with a monitoring device that poses questions regarding disease state symptoms to a person, receives answers from the person, and transmits the answers to the computer system, the method comprising:
determining whether the person should have health care professional attention based at least in part upon the answers transmitted to the computer system; and
permitting entry, storage, and presentation of intervention data.

54. The method of claim 53, wherein the intervention data includes data regarding a symptom to be counteracted and an action to be undertaken to counteract the symptom.

55. The method of claim 54, wherein the intervention data further includes the date upon which the intervention data was entered into the remote computer system.

56. The method of claim 55, wherein the intervention data further includes an indication of whether or not the action has counteracted the symptom.

57. The method of claim 53, further comprising presenting a user interface that permits viewing of a populace of persons identified as potentially needing attention by a health care professional.

58. The method of claim 53, further comprising:
presenting an operator with a set of questions, so that the operator may pose the questions to the person using the monitoring device, in response to the person having been identified as potentially needing attention by a health care professional;
wherein the set of questions are designed to permit a conclusion to be drawn regarding a diagnosis of a symptom reported by the person using the device; and
wherein the set of questions are designed to permit a conclusion to be drawn regarding selection of an intervention appropriate for the diagnosis.

59. The method of claim 58, further comprising arriving at a preliminary diagnosis and preliminary intervention as a function of the person's answers to the questions posed by the operator.

60. The method of claim 59, further comprising generating a clinical note based upon the preliminary diagnosis and the preliminary intervention.

61. The method of claim 58, wherein the set of questions is chosen based upon the answers transmitted to the remote computer by the monitoring device.

62. The method of claim 58, further comprising choosing the set of questions based upon the answers transmitted to the computer system and a measurement taken by a biometric measurement unit associated with the monitoring device.

63. The method of claim 53, further comprising automatically generating intervention data, in response to the computer system determining that the person should have health care professional attention.
